(19) United States
(12) Patent Application Publication
(10) Pub. No.: US 2011/0135704 A1
(43) Pub. Date: Jun. 9, 2011

(54) Infection Control for Surgical and Trauma Patients

(76) Inventor: John I. Shipp, Atlantic Beach, FL (US)

(21) Appl. No.: 12/764,908

(22) Filed: Apr. 21, 2010

Related U.S. Application Data
(63) Continuation-in-part of application No. 12/633,364, filed on Dec. 8, 2009.

(51) Int. Cl. A01N 63/00; A61F 2/28

(52) U.S. Cl. 424/423; 424/94.67; 623/23.6

(57) ABSTRACT

Apparatus and methods for disrupting the colonization of Staphylococcus aureus are disclosed. A lysostaphin antimicrobial coating for bone implants and a lysostaphin gel for use on open wounds to reduce infections are disclosed.

This application claims the benefit of application Ser. No. 12/633,364, filed on Dec. 8, 2009, which is hereby incorporated by reference in its entirety.

### BACKGROUND OF THE INVENTION

1. Field of the Invention

The present invention relates generally to an antimicrobial coating for bone implants and an antimicrobial gel for treating surgical and trauma wounds.

2. Technical Background

It will be appreciated by those skilled in the art lysostaphin and lysostaphin analogues are effective in killing gram positive bacteria, particularly Staphylococcus aureus and Methicillin-resistant Staphylococcus aureus (MRSA). Because of the short half-life of lysostaphin in the body, however, systemic use might require large and frequent doses to prove effective. U.S. patent application 200702924 teaches a method of extending the half-life of lysostaphin by conjugating a water soluble polymer, PEG, with lysostaphin. It is unclear, however, whether a reduction in activity that may be associated with the conjugation offsets the increase in half-life. Kokai-Kun et. al. in U.S. Pat. No. 7,572,439 ('439 patent) teaches the use of lysostaphin coated prostheses for disrupting bio-film that has formed subsequent to the initial infection.

Surgery exposes patients to infectious bacteria from sources in the operating room, including the skin and nasal passages of the staff and from the patients themselves. Surgical instruments can also be a source of bacteria. Over the past two decades, better sterilization, surgery staff preparation procedures, and the use of prophylactic antibiotics have done little to reduce infection rates. It is believed that these surgery-related infections are more frequent with larger incisions and longer lasting procedures. The reported rates of infections following laparoscopic procedures are much lower than the infection rate of comparable procedures done openly, for example.

The wounds from surgery and trauma provide an ideal growth environment for bacteria that can enter the body from the above sources. It is also well know by those skilled in the art that prosthetic implants such as hernia mesh, replacement joints, indwelling catheters, and the like provide surfaces for rapid bacteria growth. Large bacterial challenges can quickly multiply and overwhelm systemically administered antibiotics. These early onset infections most often stem from various strains of Staphylococcus aureus. Unless these large bacterial challenges are dealt with quickly, high levels of morbidity and mortality can result. Also, other bacteria, such as gram negative E. coli, can further the infection making effective treatment even more difficult. Time release antimicrobial agents and even bolus, systemically administered antibiotics are often not effective enough to contain the large challenges that are sometimes encountered. Co-pending patent application Ser. No. 12/633,364, entitled “Antimicrobial Coating for Surgical Implants and Method of Use,” teaches killing bacteria such as Staphylococcus aureus by placing an antimicrobial coating on mesh used for soft tissue repair. The teachings of that application are incorporated herein by reference in their entirety.

Infections following complex procedures such as CABG, ventral hernia repair, bariatric and orthopedic surgery are serious, painful, and expensive to treat. The infection rates following these procedures can exceed 15 percent. The problem is a huge cost to the health care system, the treatment of which can exceed the cost of the original surgery by an order of magnitude.

What is needed then is a method of lowering the infection rate following surgery by providing a quick-acting, effective quantity of antimicrobial agent at the site of the wound and at the time of the surgery or trauma that is non toxic, preferably one that is effective against large challenges of Staphylococcus aureus.

Additional features and advantages of the invention will be set forth in the detailed description which follows and, in part, will be readily apparent to those skilled in the art from that description or recognized by practicing the invention as described herein, including the detailed description which follows, the claims, and the appended drawings.

It is to be understood that both the foregoing general description and the following detailed description of the present embodiments of the invention are exemplary and explanatory, and are intended to provide an overview or framework for understanding the nature and character of the invention as it is claimed. The accompanying drawings are included to provide a further understanding of the invention and are incorporated into and constitute a part of this specification. The drawings illustrate various embodiments of the invention and, together with the description, serve to explain the principles and operations of the invention.

### BRIEF DESCRIPTION OF THE DRAWINGS

FIG. 1 shows the results of the effect of lysostaphin on spongy and cortical bone in a Staphylococcus aureus solution;

FIG. 2 illustrates the average zone of inhibition for coated versus uncoated bone;

FIG. 3 shows the PVA gel with a sample removed; and

FIG. 4 shows results of a lysostaphin gel on Staphylococcus aureus.

### DETAILED DESCRIPTION OF THE PREFERRED EMBODIMENTS

### Quantitative Kill Ratios

Those skilled in the art are aware that lysostaphin is effective in killing Staphylococcus aureus. The quantitative kill efficiency has not been widely reported however. The '439 patent teaches “administering prophylactically effective amount of an antibacterial enzyme that is lethal or damaging to a bio-film forming bacteria” but leaves to others the discovery of what an effective amount might be. The '439 patent further states “Even the most minimal concentration of enzyme will confer some protection.” In fact, the rate of kill from an antibacterial agent plus the rate of kill by the body's immune system must exceed the rate of growth of the bacteria, thus knowledge of the quantitative kill rate of the agent is critical. The size of the initial bacterial challenge is also an important factor in determining the prophylactic quantity of antibacterial agent that must be delivered to the surgical site. The most minimal concentration might have little or no clinical effect. To design an antimicrobial coating protocol for a prosthetic implant, one must decide what magnitude of bacterial challenge may be encountered under typical conditions and determine the quantity of the antibacterial agent required to kill the challenge, given the rate of growth of the bacteria and the rate of kill of the agent. Different prostheses materials require different coating protocols to attach a specific quantity of lysostaphin owing to different micro-surface areas and different affinities for adsorption of the coating. To be practical, the quantity of an antimicrobial agent required to contain a large bacterial challenge must be low compared with any toxicity limits that may exist. Experimentally determined values of Minimum Inhibitory Concentration (MIC) of an antimicrobial agent against a particular strain of bacteria can be used to estimate the number of antimicrobial molecules that is required to inhibit a given number of colony units of bacteria. Likewise bacterial growth rate can be estimated from animal model data and antimicrobial agent kill rate of the bacteria can be measured in vitro, thus allowing reasonable estimates of coating requirements of the agent on implant prostheses to assure adequate protection against surgical or trauma induced infections.

In surgical and trauma cases where there is no prosthesis to attach an antimicrobial agent, it is desirable to provide an implementation and method of use for delivering a direct, effective quantity of agent to a wound such as the open sternum in CABG procedures, C-sections, bariatric surgery, abdominal surgery, and trauma wounds. Dispersing an effective amount of lysostaphin in a cream or gel that can be directly applied to a wound offers a method of protecting against infections and is taught herein.

Antimicrobial agents bound to prostheses devices can leach and become free swimming mobile molecules in body fluids surrounding the implant and wound as taught in co-pending U.S. patent application Ser. No. 12/633,364 (the '364 application). Staphylococcus aureus can migrate over a surface by a mechanism called colony spreading as described by Kaito, et. al, Journal of Bacteriology, March 2007, p. 2553-2557, Vol. 189, No. 6. For lysostaphin to kill Staphylococcus aureus it must come into contact with the bacteria. The bacteria can spread over a prosthesis coated with lysostaphin resulting in the kill, or leached lysostaphin molecules can come into contact with bacteria attached to the surface via free swimming molecular collisions. As taught in the '364 application, both methods are effective. The '364 application also discloses the in-vivo kill rates of a 10̂7 challenge of Staphylococcus aureus by optical density activity assay for a polypropylene mesh incubated in various concentrations of lysostaphin solutions, which is reproduced in Table 1 below:

| | Initial coating | | Kill |
|---|---|---|---|
| | concentration | Change in | Rate |
| Sample | (μg/mL) | OD/hr | CFUs/hr |
| L-10 | 10 | 0.131 ± 0.008 | 1.3 × 10\hat{ }6 |
| L-25 | 25 | 0.11 ± 0.017 | 1.1 × 10\hat{ }6 |
| L-50 | 50 | 0.237 ± 0.113 | 2.4 × 10\hat{ }6 |
| L-100 | 100 | 0.187 ± 0.018 | 1.9 × 10\hat{ }6 |
| L-250 | 250 | 0.309 ± 0.096 | 3.0 × 10\hat{ }6 |
| L-500 | 500 | 0.278 ± 0.072 | 2.8 × 10\hat{ }6 |
| Uncoated mesh (control) | 0 | 0.109 ± 0.007 | 1.1 × 10\hat{ }6 |

At a concentration of 100 micrograms/ml, Table 1 above indicates a kill rate of 1.9×10̂6 CFUs/hr, for example.

### In Vivo Staphylococcus aureus Growth Rate in Rat Model with Mesh Implants

Three of 3×3 cm mesh samples, including an allograft, a xenograft, and a light-weight polypropylene mesh, were challenged with 5×10̂5 CFUs of Staphylococcus aureus after subcutaneous implant in rat models. The samples were prepared as follows.

A synthetic mesh, Ultrapro, a light-weight polypropylene mesh manufactured by Ethicon, Inc., was cut into 1×1 cm pieces in a laminar flow hood under sterile conditions prior to adsorption of an enzyme. Initial enzyme concentrations of 10, 25, 50, 100, 250, and 500 micrograms/ml in PBS buffer were prepared from a 1 mg/ml stock solution of Alexa Fluor 594-labeled lysostaphin. The initial fluorescence intensities of the enzyme sample solutions (1 mL) were measured in a 12 well plate using a microplate reader (Ex 594 nm; Em 625 nm). The samples were then added to 25 ml sterile glass vials. Using a pair of sterile tweezers, mesh pieces were gently placed into each of the vials containing the enzyme solutions and incubated overnight at room temperature with gentle shaking (100 rpm). The enzyme solution over the mesh was then collected and stored for fluorescence measurements, and the mesh was gently washed 2 times with 1 ml of PBS buffer. The wash solution was also collected and used in determination of enzyme binding yield. To remove any loosely adsorbed enzyme, 1 ml of 0.1 (v/v %) Tween 20 solution (non ionic surfactant) was then added to each of the glass vials followed by incubation for 3 hours. This surfactant solution was also collected and used in the determination of the amount of desorbed enzyme, and the mesh samples were then washed with copious amount of PBS buffer. The concentration of unbound enzyme in each of the supernatants and wash solutions was determined from fluorescence measurements. Initial enzyme solutions with known concentrations were used as the standards. The concentration of the unbound/desorbed enzyme at each step was then calculated and subtracted from initial concentration of enzyme present in the initial solution.

One square centimeter samples of an allograft mesh material Alloderm, manufactured by LifeCell Corporation, were incubated in a lysostaphin solution as described below.

The allograft material was cut into 1×1 cm pieces in a laminar flow hood under sterile conditions prior to physical adsorption after the samples were soaked in PBS buffer for rehydration.

The samples pieces were then placed in a sterile 50 ml conical tube and incubated in 30 ml PBS buffer (10 mM phosphate; 140 mM NaCl, 3 mM KCl; pH-7.4) at room temperature (RT) for 30 minutes. As per manufacturer's instructions (Calbiochem—Cat# 524650) dissolving one PBS tablet in 1 liter of deionized Water yields 140 mM NaCl, 10 mM phosphate buffer, and 3 mM KCl, pH 7.4.

The solution was then discarded and the samples were then gently flushed several times with PBS buffer using a 5 ml pipette.

The samples were incubated in 30 ml of PBS buffer at room temperature for 30 minutes and the flushing step was repeated.

The lysostaphin (Sigma Aldrich—L7386; lyophilized powder—5 mg, Protein ˜50-70%; remaining NaCl) was re-suspended in 1 ml of sterile PBS.

Initial lysostaphin concentrations of 25, 50, 100, 250, and 500 micrograms/ml PBS buffer were prepared from a 1 mg/ml stock solution of Lysostaphin.

One ml of the protein samples was then added to sterile 25 ml glass vials in a laminar flow hood under sterile conditions.

The allograft mesh material samples were gently placed into each of the vials containing the protein solutions and incubated overnight at room temperature (preferably in an incubator/shaker).

The sample solution was removed after overnight incubation and then gently flushed several times with PBS buffer using a 1 ml pipette.

The samples were then stored prior to use at 4° C. in 1 ml of PBS buffer.

Binding yield was calculated based on the amount of fluorescently labeled enzyme adsorbed on mesh and corresponded to the difference in fluorescence intensities of initial enzyme and the supernatant solutions as described above.

A xenograft mesh, Strattice manufactured by Life Cell Corporation, was soaked in sterile PBS buffer for 2 minutes according to manufacturer's instructions.

Buffer preparation: (10 mM phosphate; 140 mM NaCl, 3 mM KCl; pH-7.4) at Room Temperature for 5 min. as per manufacturer's instructions (Calbiochem—Cat# 524650). One PBS tablet was dissolved in 1 liter of deionized water to yield 140 mM NaCl, 10 mM phosphate buffer, and 3 mM KCl, pH 7.4.

The mesh was cut into 2×2 cm pieces in a laminar flow hood under sterile conditions, with the average weight of a 2×2 cm mesh being 1.25 g.

The mesh pieces were then placed in a 60 ml sterile wide mouth glass jar.

Lysostaphin solutions with concentrations of 1 and 100 micrograms/ml were prepared in PBS buffer from a 1 mg/ml stock solution of lysostaphin.

Two (2) ml of lysostaphin solution with concentrations of 1 and 100 micrograms/ml was then added to each of the glass jars containing the mesh samples in a laminar flow hood under sterile conditions and incubated for one hour at room temperature (preferably in an incubator/shaker). The lysostaphin solution was discarded and the samples were washed three times with PBS buffer using a 5 ml pipette with 40 ml of buffer.

The challenge was inserted directly onto each of the three mesh samples prior to closing the entry wound. The sample meshes were harvested after 7 days and a bacterial count was made. Table 2 depicts the average bacterial count of the three samples in each group and the average rate of growth per hour.

TABLE 2
Bacterial Count and Growth Rate 7 days of
5 × 10\hat{ }5 CFU challenge in rat model

| | Bacterial Count, | Growth Rate, |
|---|---|---|
| Mesh Sample | CFU | CFUs/hr |
| Allograft | 5 × 10\hat{ }6 | 3 × 10\hat{ }4 |
| Xenograft | 6.5 × 10\hat{ }6 | 3.9 × 10\hat{ }4 |
| Polypropylene | 2.8 × 10\hat{ }6 | 1.6 × 10\hat{ }4 |

As can been seen in Table 2 above, the growth rate of Staph. aureus in the rat model with all three type meshes is well below the kill rate of the lysostaphin coated mesh at all concentrations for a challenge of 5×10̂5 CFUs. This implies that the amount of the coating of lysostaphin should be chosen depending only on the expected challenge. The kill rate of lysostaphin is much larger than the growth rate of the bacteria so additional margin for growth rate differences need not be considered.

### Estimation of Kill Strength (Molecules per CFU)

### Lysostaphin Versus Staphylococcus aureus from MIC Measurements

Xin-Yi Yang, et. al., J Med Microbiol 56 (2007), 71-76; DOI: 10.1099, measured the MIC₉₀ values (90% kill) by recombinant lysostaphin on 257 hospital strains of Staphylococcus aureus in vitro. The strains included both meticillin-resistant Staphylococcus aureus (MRSA) and meticillin-susceptible Staphylococcus aureus (MSSA). MIC₉₀ values ranged from 0.03 to 0.5 micrograms/ml. The highest value corresponds to 1.2×10̂9 molecules of lysostaphin per CFU. This equates to 0.5 micrograms and 25 micrograms of lysostaphin for challenges of 10̂4 and 5×10̂5 respectively. Table 3 depicts the lysostaphin bound mass coated on a light-weight polypropylene mesh (60 grams per square meter) versus lysostaphin incubation concentration using the protocol taught in the '364 application and the 90% kill level calculated from MIC₉₀ data. The mesh had a micro-surface area of 72 cm̂2 as measured by the well known BET method.

TABLE 3
Bound Lysostaphin Mass versus Total Kill of Staphylococcus aureus

| Incubation | Micrograms/ | Total of | 90% Kill level of |
|---|---|---|---|
| Concentration | cm\hat{ }2 of mesh | Micrograms | Staphylococcus |
| Micrograms/ml | (micro-area) | Lysostaphin | aureus, CFU |
| 10 | 0.0075 | 1.15 | 9.7 × 10\hat{ }3 up to |
| | | | 1.6 × 10\hat{ }5 |
| 25 | 0.015 | 2.3 | 1.9 × 10\hat{ }4 up to |
| | | | 3.3 × 10\hat{ }5 |
| 50 | 0.03 | 4.6 | 3.9 × 10\hat{ }4 up to |
| | | | 6.5 × 10\hat{ }5 |
| 100 | 0.08 | 12.3 | 1 × 10\hat{ }5 up to |
| | | | 1.7 × 10\hat{ }6 |

The spread in the last column is due to the variation in MIC₉₀ over the 257 strains. The data showed no significant difference between the MIC₉₀ values of lysostaphin on Staphylococcus aureus whether the strains were MSSA or MRSA (P=0.42).

Bone implants have been used successfully in certain types of procedures; cranio-maxillofacial, orthopedic, periodontal, and hand reconstruction. As with all implant prostheses devices infections are of major concern. The present invention teaches coating both hard and soft bone implants with an antimicrobial material in sufficient quantities to kill typical challenges of bacteria that are, in general, colonized at the time of implant. Staphylococcus aureus, a gram positive pathogen, is by far the dominate infection source following such implants at a rate of about 90-95 percent. Preliminary research suggests that gram negative pathogens that are sometimes cultured from such infections develop only after, and significantly later than, the initial Staphylococcus aureus colonization. In either respect, killing Staphylococcus aureus solves by far the dominate infection problem associated with implant surgery.

### Example 1

### Bone Adsorption Protocol

Bone pieces of approximately similar weights, exhibiting both cortical and spongy characteristics, were chosen for coating with lysostaphin (totally spongy bones were discarded).

These bone pieces were then subjected to acid etching for 4 h in 100 mL of 100 mM citric acid solution at 37° C. with mild agitation so as to soften the jagged or pointed compact bone pieces, to prevent any subsequent irritation when implanted in an animal model.

The etched bone samples were then rinsed for 5 min in DI water to remove any citric acid ions loosely adhering to the bone surface.

Following the rinsing procedure, the bone samples were placed in 15 mL centrifuge tubes containing 1 mL solution of 100 μg of lysostaphin in PBS buffer prepared from a 1 mg/mL stock solution of Alexa Fluor 594-labeled lysostaphin for 1 h, in an incubator at 37° C. under constant agitation.

Following adsorption of lysostaphin, the supernatant was collected and the samples were subject to two step washing procedure, initially with 1 mL of PBS vigorously agitated at 37° C. for 3 min (this wash was collected to determine amount of desorbed Lysostaphin) and subsequently in 3 mL of PBS for another 5 mins before transferring the sample into a sterile container containing excess of PBS buffer solution.

Following this, the supernatants of both the lysostaphin and the first wash solution were analyzed for the amount of unbound lysostaphin using BCA assay. Solutions with known lysostaphin concentrations were used as the standards.

### Turbimetric Assay of Lysostaphin Coated Bone Samples

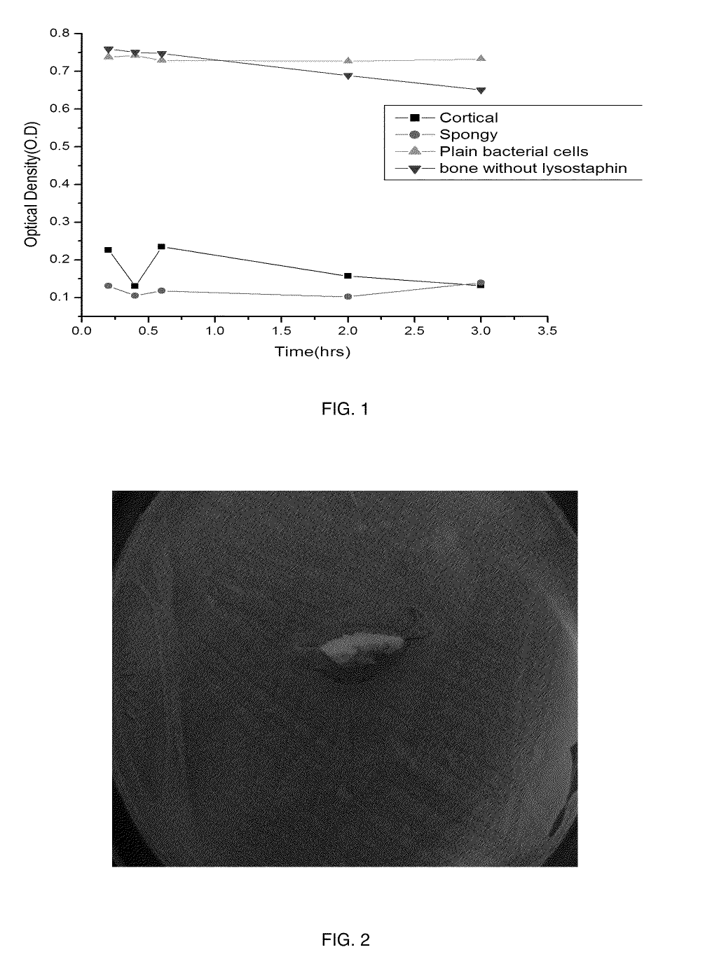

A Turbimetric assay was conducted on two bone samples, one cortical and one spongy, incubated in a solution of 100 micrograms/ml resulting in adsorption of 11.2 and 18.8 micrograms of lysostaphin, respectively. The samples were then placed in a Staphylococcus aureus solution following the protocol described in the '364 application. The optical density of the solution was measured over a 3 hour period for each of the coated samples and controls without lysostaphin coating. FIG. 1 depicts the results.

Illustrated in FIG. 1 is a rapid and effective kill of both bone types.

### Zone of Inhibition Measurements

A bone sample was incubated according to the above protocol resulting in 15 micrograms of lysostaphin coated on the sample and compared with a control sample with no coating. The samples were placed in a buffered solution containing a Staphylococcus aureus challenge of 10̂6 CFU/ml. The zone of inhibition was measured in four quadrants using a scale to measure the distance from the edges of the bone to the density demarcation lines. Table 4 depicts the measurements for the control and coated samples.

| Zone | Zone Dimension, cm | Zone Dimension, cm |
|---|---|---|
| Location | Control, No LYS | LYS Coated Sample |
| Top | 0.1 | 0.31 |
| Right | 0.1 | 0.2 |
| Bottom | 0.11 | 0.09 |
| Left | 0.22 | 0.32 |
| Average | 0.13 | 0.23 |

The average zone of inhibition for the Lysostaphin coated sample is seen to be 80 percent larger than the uncoated bone, indicating effective kill of the bacteria. The coated sample and its zone of inhibition can be seen in FIG. 2

### Method of Use-Lysostaphin Coated Bone and Bone Substitutes

Prior to surgery providing a bone and or bone substitute prosthesis incubated in a lysostaphin solution of such concentration that yields a least 0.1 micrograms per cm̂2 of micro-surface area for an expected challenge of 10̂6 CFUs of Staphylococcus aureus, implanting the prosthesis into a human body to disrupt the colonization of Staphylococcus aureus, and closing the entry wound.

### Example 2

### Antimicrobial Gel for Killing Staphylococcus aureus

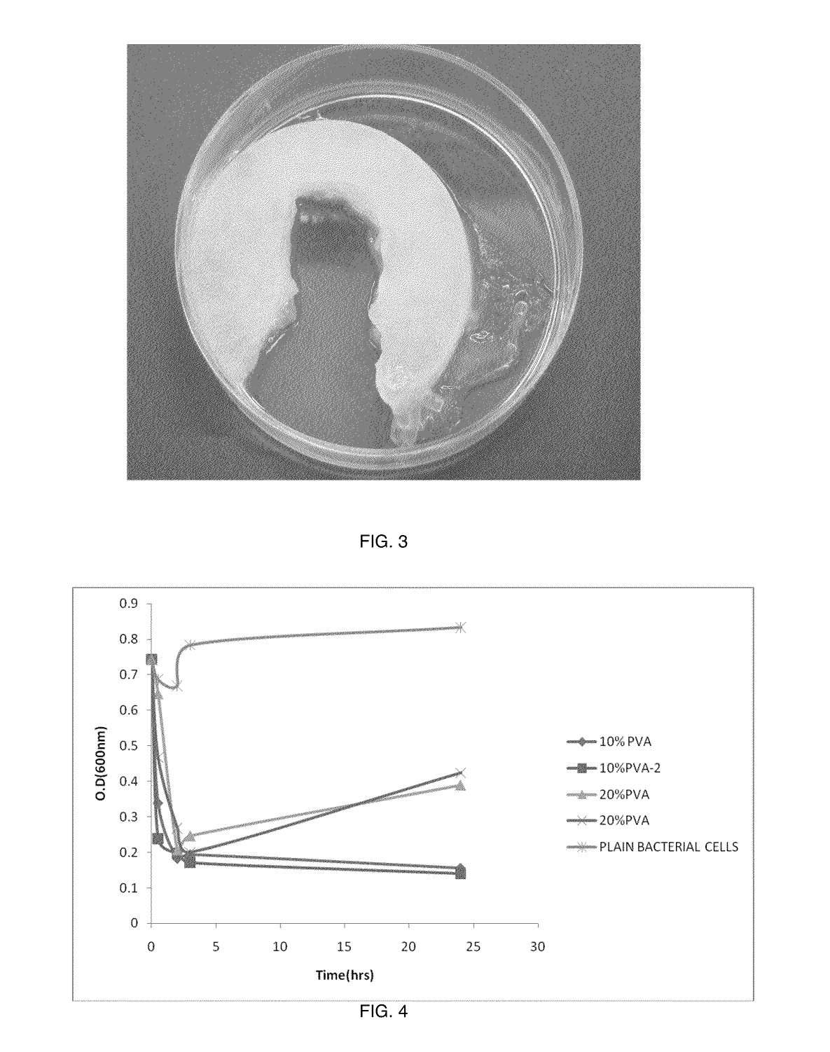

A 100 microgram/ml concentration of lysostaphin solution in PBS buffer was prepared from a 1 mg/mL stock solution of Alexa Fluor 594-labeled lysostaphin. Two samples of lysostaphin gel were prepared by dissolving 10 and 20 percent by weight of polyvinyl alcohol (PVA) in the microgram/ml concentration of lysostaphin solution. One square cm of the two gels was cut from the samples and placed into Staphylococcus aureus suspension described above and the kill rate was measured by standard turbidity methods. FIG. 3 depicts the 10% PVA gel with the sample cut out.

The Turbidity Assay is shown in FIG. 4, which shows that the 10% PVA gel displays a higher bacterial kill rate. The consistency of the gel will allow the antimicrobial loaded gel to be spread over the edges of an open surgical or trauma wound for infection control. The gel can be put through one or more freeze-dried thawing cycles for stability and increased mechanical properties due to physical crosslinking.

### Method of Use-Gel or Cream Infused with Lysostaphin

Following surgery or trauma before wound closure providing a lysostaphin infused gel prepared from at least a concentration of 100 micrograms/ml of lysostaphin and at most a 20 percent by weight of polyvinyl alcohol so as to form a gel, coating the wound edges with the infused gel to disrupt the colonization of Staphylococcus aureus, and closing the wound.

I claim:

1. A method of treating infections in a mammal comprising:
providing a bone implant having a therapeutic amount of antimicrobial properties;
implanting the bone implant at an implant location to inhibit growth of pathogens at the implant location.

2. The method according to claim 1, wherein the bone implant is cortical bone.

3. The method according to claim 1, wherein the bone implant includes spongy bone.

4. The method according to claim 1, wherein the antimicrobial properties are a result of leached and attached antimicrobial molecules.

5. A method of minimizing bacterial growth in a mammal undergoing bone implant surgery comprising:
providing a bone implant;
coating the bone implant with an antimicrobial agent;
implanting the coated bone implant through a surgical opening; and
closing the surgical opening.

6. The method according to claim 5, wherein the bone implant is cortical bone.

7. The method according to claim 5, wherein the bone implant is spongy bone.

8. The method according to claim 5, wherein the bone implant is spongy and cortical bone.

9. The method according to claim 5, wherein coating the bone implant is by physical adsorption of the antimicrobial material by the bone implant.

10. The method according to claim 5, wherein coating the bone implant is by covalent bonding of the antimicrobial material onto the bone implant.

14. The method according to claim 5, wherein the antimicrobial agent is pharmacologically effective against gram positive bacteria.

15. The method according to claim 5, wherein the antimicrobial agent is lysostaphin.

16. The method according to claim 5, wherein the antimicrobial agent is pharmacologically effective against gram positive and gram negative bacteria.

17. The method according to claim 5, further comprising coating the surgical opening with a gel infused with the antimicrobial agent prior to closing the surgical opening.

18. The method according to claim 17, wherein the infused gel is polyvinyl alcohol and lysostaphin.
